(19) United States
(12) Patent Application Publication
(10) Pub. No.: US 2025/0099759 A1
(43) Pub. Date: Mar. 27, 2025

(54) TRANSMISSION OF SIGNAL INFORMATION TO AN IMPLANTABLE MEDICAL DEVICE

(71) Applicant: Cochlear Limited, Macquarie University, NSW (AU)

(72) Inventors: Michael GOOREVICH, Naremburn, NSW (AU); Phyu Phyu KHING, North Sydney, NSW (AU); Michael John PHILLIPS, Alfords Point, NSW (AU)

(21) Appl. No.: 18/730,892

(22) PCT Filed: Jan. 11, 2023
(86) PCT No.: PCT/IB2023/050253

§ 371 Date: Jul. 22, 2024

Related U.S. Application Data
(60) Provisional application No. 63/304,014, filed on Jan. 28, 2022.

(51) Int. Cl. A61N 1/36; A61N 1/372

(52) U.S. Cl. CPC A61N 1/36038; A61N 1/37217; A61N 1/37252

(57) ABSTRACT

Presented herein are techniques for wirelessly communicating sound data and one or more sound signal attributes from an external component of a medical device to an implantable component of the medical device via one or more data packets. For example, when the medical device is embodied as a hearing device, such as a cochlear implant system or hearing aid, sensory data and one or more sensory signal attributes determined from input sensory data can be combined into data packets by an external component and wirelessly transmitted to an implantable component of the cochlear implant system. The cochlear implant can separate the sensory data and one or more sensory signal attributes from the data packets and further process the sensory data using the one or more sensory signal attributes in order to generate stimulation signals for delivery to a recipient of the cochlear implant system.

### BACKGROUND

### Field of the Invention

The present invention relates generally to implantable medical devices in which signal information is transmitted/sent to an implantable medical device.

### Related Art

Medical devices have provided a wide range of therapeutic benefits to recipients over recent decades. Medical devices can include implantable components/devices, external or wearable components/devices, or combinations thereof (e.g., a device having an external component communicating with an implantable component). Medical devices, such as traditional hearing aids, partially or fully-implantable hearing devices (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a first method is provided. The first method comprises: receiving sensory signals at an external component of an implantable medical device system that is in wireless communication with an implantable component of the implantable medical device system; converting the sensory signals to sensory data; determining at least one sensory signal attribute of the sensory signals; combining the sensory data and the at least one sensory signal attribute into one or more data packets; and sending the one or more data packets to the implantable component of the implantable hearing device system via wireless communications

In another aspect, one or more non-transitory computer readable storage media are provided. The one or more non-transitory computer readable storage media comprise instructions that, when executed by a processor, cause the processor to: receive sound signals at an external component of an implantable hearing device system that is in wireless communication with an implantable component of the implantable hearing device system; convert the sound signals to sound data; determine at least one sound signal attribute of the sound signals; combine the sound data and the at least one sound signal attribute into one or more data packets; and send the one or more data packets to the implantable component of the implantable hearing device system via wireless communications.

In another aspect, one or more non-transitory computer readable storage media are provided. The one or more non-transitory computer readable storage media comprise instructions that, when executed by a processor, cause the processor to: convert sound signals received at an external component of an implantable hearing device system to sound data; determine at least one sound signal attribute of the sound signals; and stream one or more data packets to the implantable component of the implantable hearing device system via wireless communications, wherein at least one data packet of the one or more data packets comprises the sound data and the at least one sound signal attribute.

In another aspect, an implantable hearing device system is provided. The implantable hearing device system comprises: one or more microphones; and one or more processors, wherein the one or more processors are configured to: receive sound signals at an external component of an implantable hearing device system that is in wireless communication with an implantable component of the implantable hearing device system; convert the sound signals to sound data; determine at least one sound signal attribute of the sound signals; combine the sound data and the at least one sound signal attribute into one or more data packets; and send the one or more data packets to the implantable component of the implantable hearing device system via wireless communications.

In another aspect, an implantable hearing device system is provided. The implantable hearing device system comprises an external component comprising: one or more input devices; a wireless transceiver; and one or more processors, wherein the one or more processors are configured to: convert received sound signals received at the one or more input devices to sound data; determine at least one sound signal attribute of the sound signals; and stream one or more data packets to an implantable component of the implantable hearing device system via wireless communications, wherein at least one data packet of the one or more data packets comprises the sound data and the at least one sound signal attribute.

In another aspect, a second method is provided. The second method comprises: receiving one or more data packets by an implantable component of an implantable medical device system from an external component of the implantable hearing device system via wireless communications, wherein at least one data packet comprises sensory data and at least one sensory signal attribute; separating the sensory data and the at least one sensory signal attribute from the at least one data packet; and processing the sensory data utilizing the at least one sensory signal attribute to generate stimulation control signals for use in stimulating a recipient of the implantable medical device system.

In another aspect, one or more non-transitory computer readable storage media are provided. The one or more non-transitory computer readable storage media comprise instructions that, when executed by a processor, cause the processor to: receive one or more data packets by an implantable component of an implantable hearing device system from an external component of the implantable hearing device system via wireless communications, wherein at least one data packet comprises sound data and at least one sound signal attribute; separate the sound data and the at least one sound signal attribute from the at least one data packet; and process the sound data utilizing the at least one sound signal attribute to generate stimulation control signals for use in stimulating a recipient of the implantable hearing device system.

In another aspect, an implantable hearing device system is provided. The implantable hearing device system comprises: one or more microphones; and one or more processors, wherein the one or more processors are configured to: receive one or more data packets by an implantable component of an implantable hearing device system from an external component of the implantable hearing device system via wireless communications, wherein at least one data packet comprises sound data and at least one sound signal attribute; separate the sound data and the at least one sound signal attribute from the at least one data packet; and process the sound data utilizing the at least one sound signal attribute to generate stimulation control signals for use in stimulating a recipient of the implantable hearing device system.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A; FIG. 1D is a block diagram of the cochlear implant system of FIG. 1A;

FIG. 2A is a functional block diagram illustrating further details of an external component of a cochlear implant system configured to implement certain techniques presented herein;

FIG. 2B is a functional block diagram illustrating further details of an implantable component of the cochlear implant system of FIG. 2A configured to implement certain techniques present herein;

FIG. 3A is a functional block diagram illustrating further details of an external component of a cochlear implant system configured to implement certain techniques presented herein;

FIG. 3B is a functional block diagram illustrating further details of an implantable component of the cochlear implant system of FIG. 3A configured to implement certain techniques present herein;

FIG. 4 is a schematic diagram illustrating an example packet structure that may be utilized to carry signal data and signal attribute information to implement certain techniques presented herein;

FIG. 5 is a flowchart illustrating a first example process for providing external component to implant component transmissions including signal attribute information;

FIG. 6 is a flowchart illustrating a second example process for providing external component to implant component transmissions including signal attribute information; and

FIG. 7 is a functional block diagram of an implantable stimulator system with which aspects of the techniques presented herein can be implemented.

### DETAILED DESCRIPTION

Certain implantable medical device systems, such as implantable auditory prostheses, include both an implantable component and an external component. The external component can be configured to capture environmental signals (e.g., sensory or sound signals) and transmit/send the environmental signals (e.g., audio data), or a processed version thereof (e.g., stimulation control signal data), to the implantable component. Raw/captured environmental signals (e.g., sensor or sound signals) and processed environmental signals (e.g., stimulation control signal data or processed audio) are collectively and generally referred to herein as “signal data” or, in the specific context of auditory prostheses, interchangeably referred to herein as “sensory data” or “sound data.” Presented herein are techniques for combining the signal data (e.g., sensory or sound data) with “signal attribute information” (e.g., sensory or sound signal attributes extracted from the raw/captured environmental signals) for wireless transmission from an external component to an implantable component.

For example, when a medical device is embodied as a hearing device, such as a cochlear implant system or other auditory prosthesis, sensory or sound signals can be received by an external component. The external component is configured to analyze the sensory/sound signals to extract signal attribute information from the sensory/sounds signals. The external component is configured to wirelessly send/transmit the signal attribute information and signal data (e.g., audio data or stimulation control signal data) to the implantable component (e.g., via one or more wireless packets in which one or more of the packets can include the signal attribute information that has been extracted or determined from the received sensory/sound signals).

The techniques presented herein may be beneficial for a number of different medical device recipients. In one instance, techniques presented herein may help to avoid the need to implement complicated tasks within an implantable component, which can help to save power consumption by the implantable component. For example, techniques presented herein can minimize or eliminate the need to perform complicated calculations for audio feature/signal attribute extraction by an implantable component by pushing such operations to an external component that can more easily calculate such features, referred to herein as “signal attributes” (e.g., sound signal attributes) and then provide this information, along with matching signal data, which can reduce power consumption by the implantable component. As noted, in some instances, processing performed by the external component can involve generating stimulation control signal data that can be sent to an implantable component along with sound signal attributes, which may provide further power savings for the implantable component.

Merely for ease of description, the techniques presented herein are primarily described with reference to a specific medical device system, namely a cochlear implant system. However, it is to be appreciated that the techniques presented herein may also be partially or fully implemented by other types of implantable medical device systems. For example, the techniques presented herein may be implemented by other auditory prosthesis or hearing device systems, such as hearing aids, middle ear auditory prostheses, bone conduction devices, direct acoustic stimulators, electro-acoustic prostheses, auditory brain stimulators, combinations or variations thereof, etc. The techniques presented herein may also be implemented by dedicated tinnitus therapy devices and tinnitus therapy device systems. In further embodiments, the presented herein may also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc. A cochlear implant system can be referred to interchangeably herein as an implantable hearing device system.

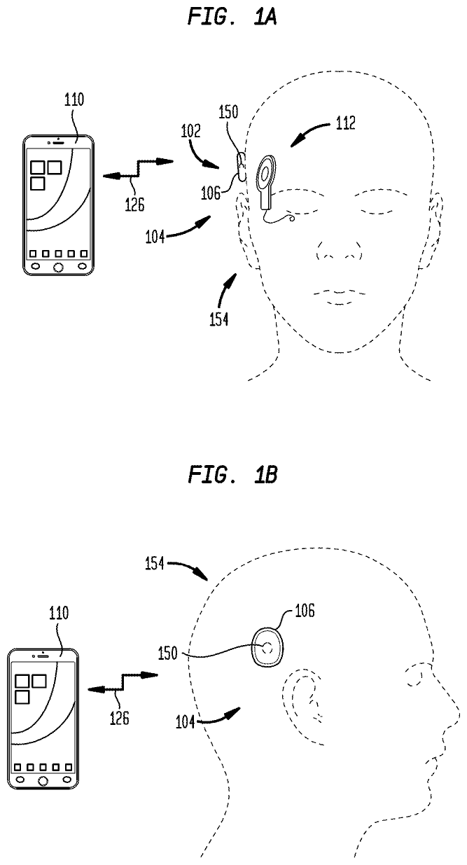

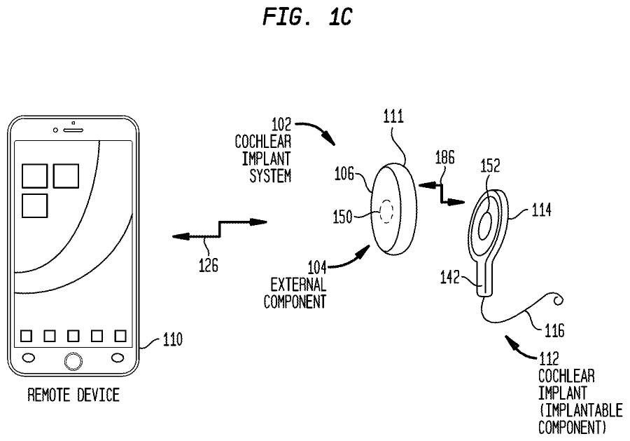

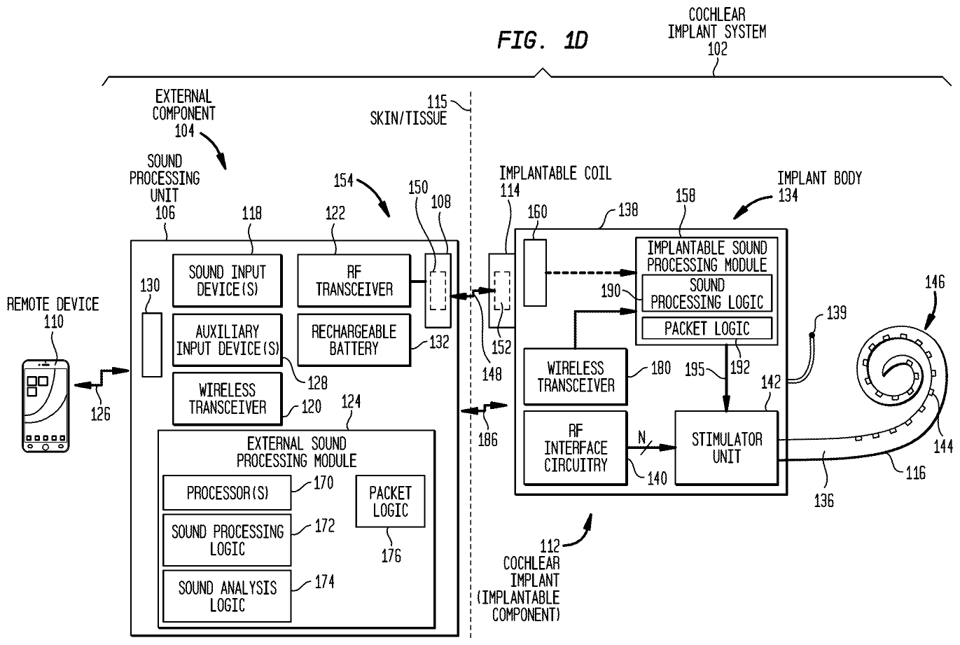

FIGS. 1A-1D illustrates an example cochlear implant system 102 with which aspects of the techniques presented herein can be implemented. The cochlear implant system 102 comprises an external component 104 and an implantable component 112. In the examples of FIGS. 1A-1D, the implantable component 112 is sometimes referred to as a “cochlear implant 112.” FIG. 1A illustrates the cochlear implant 112 implanted in the head 154 of a recipient, while FIG. 1B is a schematic drawing of the external component 104 worn on the head 154 of the recipient. FIG. 1C is another schematic view of the cochlear implant system 102, while FIG. 1D illustrates further details of the cochlear implant system 102. For ease of description, FIGS. 1A-1D will generally be described together.

Cochlear implant system 102 includes an external component 104 that is configured to be directly or indirectly attached to the body of the recipient and an implantable component 112 configured to be implanted in the recipient. In the examples of FIGS. 1A-1D, the external component 104 comprises a sound processing unit 106, while the cochlear implant 112 includes an implantable coil 114, an implant body 134, and an elongate stimulating assembly 116 configured to be implanted in the recipient's cochlea.

In the example of FIGS. 1A-1D, the sound processing unit 106 is an off-the-ear (OTE) sound processing unit, sometimes referred to herein as an OTE component, that is configured to send data and power to the implantable component 112. In general, an OTE sound processing unit is a component having a generally cylindrically shaped housing 111 and which is configured to be magnetically coupled to the recipient's head (e.g., includes an integrated external magnet 150 configured to be magnetically coupled to an implantable magnet 152 in the implantable component 112). The OTE sound processing unit 106 also includes an integrated external (headpiece) coil 108 that is configured to be inductively coupled to the implantable coil 114.

It is to be appreciated that the OTE sound processing unit 106 is merely illustrative of the external devices that could operate with implantable component 112. For example, in alternative examples, the external component may comprise a behind-the-ear (BTE) sound processing unit or a micro-BTE sound processing unit and a separate external. In general, a BTE sound processing unit comprises a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external components could be located in the recipient's ear canal, worn on the body, etc.

As noted above, the cochlear implant system 102 includes the sound processing unit 106 and the cochlear implant 112. However, as described further below, the cochlear implant 112 can operate independently from the sound processing unit 106, for at least a period, to stimulate the recipient. For example, the cochlear implant 112 can operate in a first general mode, sometimes referred to as an “external hearing mode,” in which the sound processing unit 106 captures sensory/sound signals which are then used as the basis for delivering “sensory data” or “sound data,” such as audio signal data or stimulation control signal data (stimulation data), to the cochlear implant through which electrical stimulation signals can further be generated by the cochlear implant 112 and delivered to the recipient. The cochlear implant 112 can also operate in a second general mode, sometimes referred as an “invisible hearing” mode, in which the sound processing unit 106 is unable to provide sensory/sound data to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sensory/sound signals itself via implantable sound sensors and then uses those sound signals as the basis for delivering stimulation signals to the recipient. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with a remote device 110 that can, in some embodiments, be configured to implement aspects of the techniques presented. The remote device 110 is a computing device, such as a computer (e.g., laptop, desktop, tablet), a mobile phone, remote control unit, etc. Sound processing unit 106 includes a wireless transmitter/receiver (transceiver) 120. The remote device 110 and the cochlear implant system 102 (e.g., OTE sound processing unit 106 or, in some instances, the cochlear implant 112) can wirelessly communicate via a bi-directional wireless communication link 126. In accordance with techniques presented herein, sound processing unit 106 and implantable component 112 may also wirelessly communicate via a bi-directional wireless communication link 186. Each bi-directional wireless communication link 126 and 186 may comprise, for example, a short-range communication interface, such as Bluetooth link, Bluetooth Low Energy (BLE) link, a proprietary communication interface, or other communication interface making use of any number of standard wireless streaming protocols that may utilize any scheme of wireless communication channels, radio frequencies, etc. in order to facilitate wireless communications involving one or more packets communicated between various components (e.g., between remote device 110 and sound processing unit 106 via wireless communication link 126 and/or between sound processing unit 106 and implantable component 112 via wireless communication link 186. Bluetooth® is a registered trademark owned by the Bluetooth® SIG.

Returning to the example of FIGS. 1A-1D, the OTE sound processing unit 106 comprises one or more input devices that are configured to receive input signals (e.g., sensory/sound or data signals). The one or more input devices include one or more sound input devices 118 (e.g., one or more external microphones, audio input ports, telecoils, etc.).

According to the techniques of the present disclosure, sound input devices 118 may include two or more microphones or at least one directional microphone. Through such microphones, directionality of the microphones may be optimized, such as optimization on a horizontal plane defined by the microphones. Accordingly, classic beamformer design may be used for optimization around a polar plot corresponding to the horizontal plane defined by the microphone(s).

Also included in the sound processing unit 106 are one or more auxiliary input devices 128 (e.g., audio ports, such as a Direct Audio Input (DAI), data ports, such as a Universal Serial Bus (USB) port, cable port, etc.). However, it is to be appreciated that one or more input devices may include additional types of input devices and/or less input devices (e.g., one or more auxiliary input devices 128 could be omitted).

The OTE sound processing unit 106 also comprises the external coil 108, a charging coil 130, a closely-coupled transmitter/receiver 122, sometimes referred to as a radio-frequency (RF) transceiver 122, at least one rechargeable battery 132, and an external sound processing module 124. The external sound processing module 124 may comprise, for example, one or more processors and a memory device (memory) that includes one or more processors 170 (e.g., one or more Digital Signal Processors (DSPs), one or more microcontroller cores, one or more hardware processors, etc.) and a number of logic elements, such as sound processing logic 172, sound analysis logic 174, and packet logic 176. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in the memory device.

As discussed in further detail herein, packet logic 176 facilitates sound data streaming to the implantable component by performing packet encoding or mapping operations involving combining sensory/sound data, such as audio signal data or stimulation control signal data (determined from input sound signals), along with at least one sensory/sound signal attribute, into one or more data packets 188 that can be wirelessly transmitted to implantable component 112. In some instances, packet logic 176 may also perform packet decoding or de-mapping operations for any packets that may be received by sound processing unit 106, for example, for packets that may be received by or otherwise streamed to sound processing unit 106 from remote device 110 or that may be received from implantable component 112, such as acknowledgments (ACKs) regarding packets transmitted from sound processing unit 106 to implantable component 112 and/or for any requests for data/information that may be generated by implantable component 112 and sent to sound processing unit 106 (e.g., configuration data, firmware updates, etc.).

The implantable component 112 comprises an implant body (main module) 134, a lead region 136, and the intra-cochlear stimulating assembly 116, all configured to be implanted under the skin/tissue (tissue) 115 of the recipient. The implant body 134 generally comprises a hermetically-sealed housing 138 in which RF interface circuitry 140 and a stimulator unit 142 are disposed. The implant body 134 further includes a wireless transceiver 180 that facilitate wireless communications for the implantable component 112. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the recipient's cochlea. Stimulating assembly 116 includes a plurality of longitudinally spaced intra-cochlear electrical stimulating contacts (electrodes) 144 that collectively form a contact or electrode array 146 for delivery of electrical stimulation (current) to the recipient's cochlea.

Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 152 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, may be used to transfer the power and/or data from an external component to an implantable component and, as such, FIG. 1D illustrates only one example arrangement.

As noted above, sound processing unit 106 includes the external sound processing module 124. The external sound processing module 124 is configured to convert received input sound signals (sensory/sound received at one or more of the input devices) into output signals for use in stimulating a first ear of a recipient (i.e., the external sound processing module 124 is configured to perform sound processing on input sound signals received at the sound processing unit 106). Stated differently, the one or more processors 170 in the external sound processing module 124 are configured to execute sound processing logic 172 in memory to convert the received input sound signals into output sound data, such as audio signal data or stimulation control signal data, that can be used by the implantable component 112 to generate electrical stimulation for delivery to the recipient.

In one embodiment, the external sound processing module 124 in the sound processing unit 106 can perform extensive sound processing operations to generate output sound data that is inclusive of stimulation control signal data. In an alternative embodiment, the sound processing unit 106 can send less processed information, such as audio signal data, to the implantable component 112 and the sound processing operations (e.g., conversion of sounds to output stimulation signals) can be performed by a processor within the implantable component 112.

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the recipient's auditory nerve cells. Additionally, in accordance with embodiments herein, varying levels of sound processing operations can be performed by the cochlear implant 112, depending on the type of sensory/sound data (i.e., audio signal data or stimulation control signal data) that is wirelessly transmitted from the external component 104 (via sound processing unit 106/wireless transceiver 120) to the implantable component 112.

As shown in FIG. 1D, the cochlear implant 112 includes a plurality of implantable sound sensors 160 and an implantable sound processing module 158. Similar to the external sound processing module 124, the implantable sound processing module 158 may comprise, for example, one or more processors (not shown) and a memory device (memory) that includes sound processing logic 190 and also packet logic 192. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device. Regarding wireless communications provided for the implantable component 112 via wireless transceiver 180 and packet logic 192, for one or more packets received by the implantable component 112 from sound processing unit 106, packet logic 192 can decode or de-map the one or more packets in order to generate the audio signal data or stimulation control signal data included in the packets and, if present in one or more received packets, can also decode, de-map, or otherwise separate one or more sensory/sound signal attributes from the packets for further processing the audio signal data or the stimulation control signal data by the implantable component 112 based using the sensory/sound signal attributes in order to generate stimulation signals for delivery to the recipient.

For completeness, it is noted that external sound processing module 124 may be embodied as a BTE sound processing module or an OTE sound processing module. Accordingly, the techniques of the present disclosure are applicable to both BTE and OTE hearing devices.

Conventionally, in the invisible hearing mode, the implantable sound sensors 160 are configured to detect/capture signals (e.g., acoustic sensory/sound signals, vibrations, etc.), which are provided to the implantable sound processing module 158. The implantable sound processing module 158 is configured to convert received input signals (received at one or more of the implantable sound sensors 160) into stimulation control signals 195 for use in stimulating the first ear of a recipient (i.e., the processing module 158 is configured to perform sound processing operations). Stated differently, the one or more processors in implantable sound processing module 158 are configured to execute sound processing logic in memory to convert the received input signals into stimulation control signals 195 that are provided to the stimulator unit 142. The stimulator unit 142 is configured to utilize the stimulation control signals 195 to generate electrical stimulation signals (e.g., current signals) for delivery to the recipient's cochlea, thereby bypassing the absent or defective hair cells that normally transduce acoustic vibrations into neural activity. The terms “stimulation control signal data” and “stimulation control signals” are both utilized herein to refer to signals that represent electrical stimulation that can be delivered to the recipient. However, in the context of embodiments herein, stimulation control signal data represents data generated by the external component 104 that can be further processed by the implantable component 112 utilizing sensory/sound signal attributes received from the external component 104 in order to generate stimulation control signals 195 that are provided to the stimulator unit 142, through which electrical stimulation signals are generated for delivery to the recipient.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 160 in generating stimulation signals for delivery to the recipient.

As noted above, implantable medical devices, such as cochlear implant system 102 of FIG. 1D, may include microphones that operate according to operational parameters that allow the microphones to operate with directionality to improve signal-to-noise ratio (“SNR”) of the processed audio signals. This microphone directionality allows recipients to have, for example, improved speech recognition in noisy situations. These microphone directionality techniques rely on the user facing the speaker so the directional microphones may pick up the speaker's voice and block out noise to the sides and rear of listener.

In addition to the conventional external hearing mode and invisible hearing mode, in accordance with techniques presented herein, as noted above, a wireless interface can further be defined to facilitate sending stimulation control signal data or audio signal data from an external component to an implantable component of an implantable medical device. Broadly, in the context of cochlear implant system 102, the external component 104, via sound processing unit 106, can convert input sound signals to sound data, such as audio signal data or stimulation control signal data, and can also determine one or more sound signal attributes from the input sound signal data. The sound data and one or more sound signal attributes can be combined into one or more packets, via packet logic 176, and wirelessly transmitted (via wireless transceiver 120) to the implantable component 112. The implantable component 112 can receive the one or more packets via wireless transceiver 180 and, via packet logic 182, can decode or de-map the sound data and, if present, separate the one or more sound signal attributes from the one or more packets and deliver the sound data and the sound signal attributes to implantable sound processing module 158, which can generate output stimulation signals that can be delivered to the recipient via the intra-cochlear stimulating assembly 116.

In the case of the external component sending stimulation control signal data, various sensory/sound signal attributes can be extracted from the sensory/sound signals and included with the stimulation control signal data sent to the implantable component 112. For example, cochlear implant coding strategies, such as the Optimized Pitch and Language (OPAL) coding/processing strategy, utilize the extraction of a fundamental frequency (F0) estimate along with a sound signal, which may be better suited to the processing capabilities of the external component 104, and could even be done off-line (e.g., when streaming sound from a smart phone or tablet, such as from remote device 110). However, when sending audio signal data to an implantable component 112, it is likely that further processing on the implantable component may be kept to a minimum, in order to reduce power consumption.

Accordingly, techniques as further described herein below allow a sound data stream, such as an audio signal or stimulation control signal data stream, along with markers or sound signal attributes that can be correlated with the data stream, such as F0 estimates, Periodic Probability Estimate (PPE) signal attributes, environmental classifier data, and/or any other sound signal attributes, to be wirelessly transmitted from the external component 104 to the implantable component 112, which can be used by the implantable component 112 to generate stimulation control signals that are delivered to the recipient.

One benefit of this approach is that implantable component 112 processing can be minimized, while supporting an implant audio interface and coding strategies such as OPAL. The concept can be expanded to include other sound signal attributes such as harmonic probabilities, Automatic Gain Control (AGC) levels, adaptive filter states, signal level or features that can be utilized by an environmental classifier operating in the implantable component 112.

Techniques presented herein may also be extended to use cases in which music and/or speech is being transmitted by a mobile assistive device to an external component and, in case OPAL is the preferred sound coding strategy, features/signal attribute information sent by the mobile device to the external component can use this information for sound processing performed on the music/speech received from the mobile assistive device.

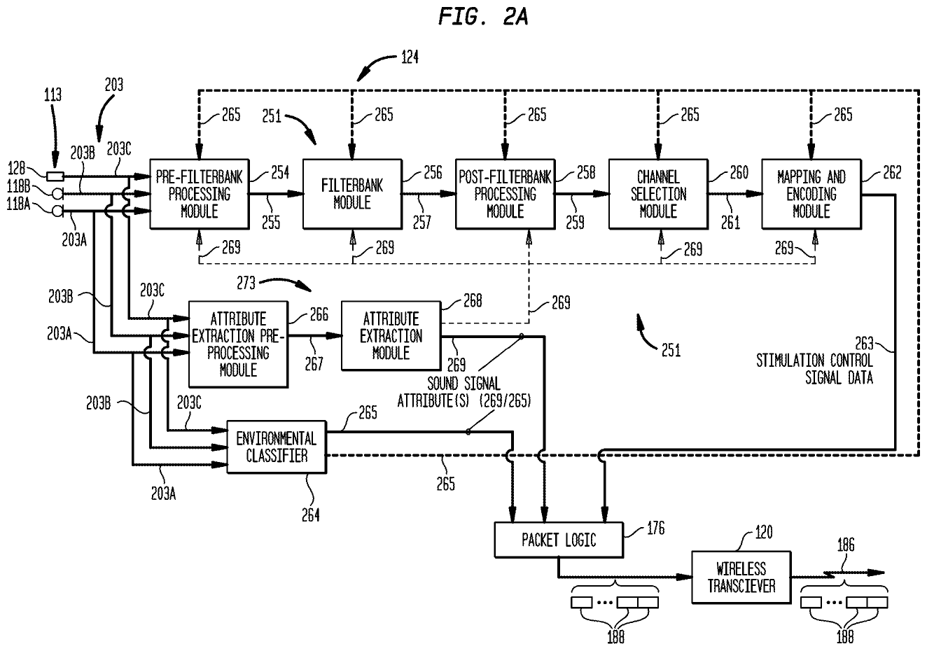

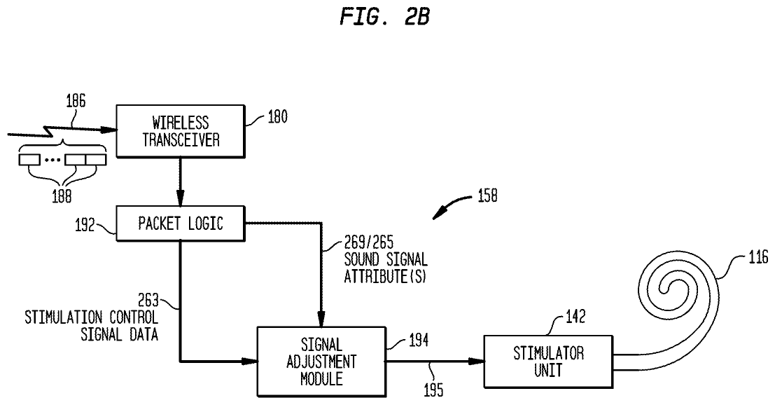

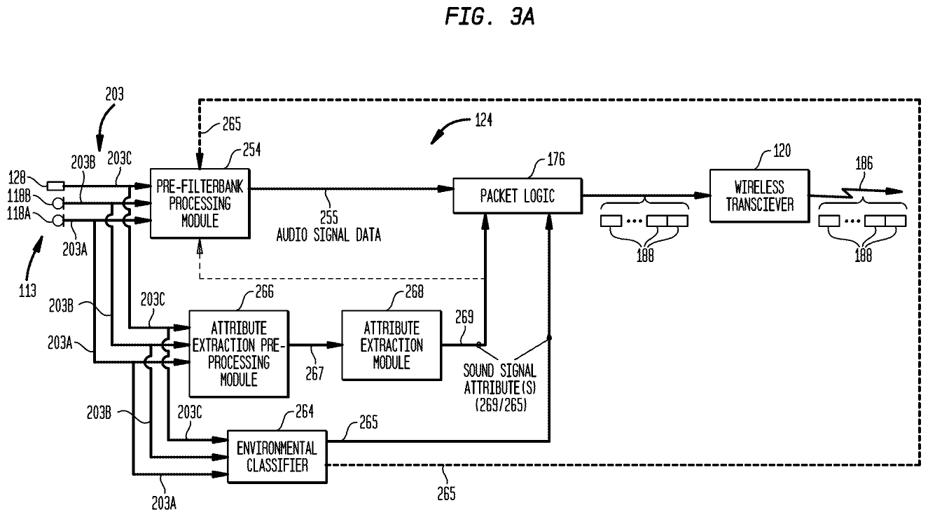

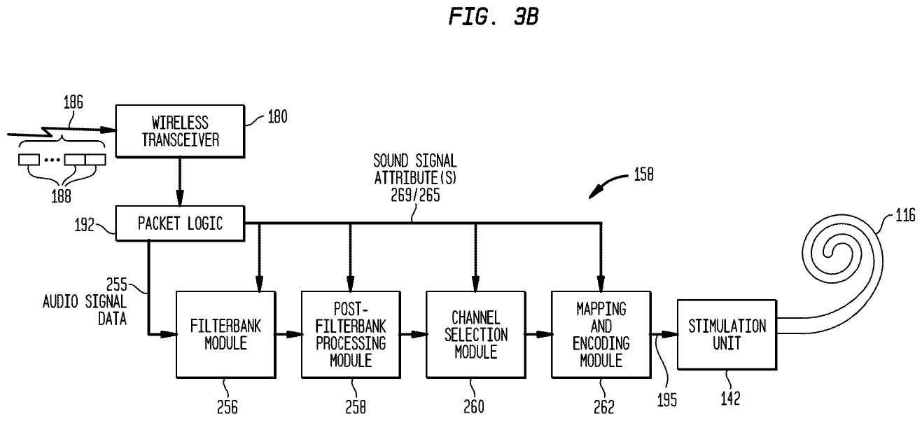

Consider various operational examples, as discussed in further detail below with reference to FIGS. 2A-2B and FIGS. 3A-3B. In accordance with techniques of this disclosure, any externally calculated/extracted sensory/sound signal attributes and/or signal path parameters/attributes could be calculated and/or extracted from electrical sensory/sound signals received by the external component and/or via a sensory/sound processing path for the external component 104 and passed to the implantable component 112 as sensory/sound signal attributes carried in packets with sensory/sound data.

With reference to FIG. 2A, FIG. 2A is a functional block diagram illustrating further details of the sound processing unit 106 of cochlear implant system 102 configured to implement certain techniques in which the sound processing unit 106, via external sound processing module 124, generates stimulation control signal data 263 and one or more sensory/sound signal attributes (e.g., sound signal attributes 269/265) that are combined into data packets 188 and transmitted to the implantable component 112 of FIG. 2B via wireless communication link 186, in accordance with an embodiment.

As noted, the external component 104 comprises one or more input devices, labeled as input devices 113 in FIG. 2A. In the example of FIG. 2A, the input devices 113 comprise two sound input devices, namely a first microphone 118A and a second microphone 118B, as well as at least one auxiliary input device 128 (e.g., an audio input port, a cable port, a telecoil, etc.). If not already in an electrical form, input devices 113 convert received/input sound signals into electrical sound signals 203, referred to herein as electrical sound signals, which represent the sound signals received at the input devices 113. As shown in FIG. 2A, the electrical sound signals 203 include electrical sound signal 203A from microphone 118A, electrical sound signal 203B from microphone 118B, and electrical sound signal 203C from auxiliary input 128.

Also as noted above, the external component 104 comprises the external sound processing module 124 which, for the embodiment of FIG. 2A includes, among other elements, one or more processors 170, sound processing logic 172 and sound analysis logic 174. The sound processing logic 172, when executed by the one or more processors 170, enables the external sound processing module 124 to perform sound processing operations that convert sound signals into stimulation control signal data 263 for use in delivery of stimulation to the recipient. In FIG. 2A, the functional operations enabled by the sound processing logic 172 (i.e., the operations performed by the one or more processors 170 when executing the sound processing logic 172) are generally represented by modules 254, 256, 258, 260, and 262, which collectively comprise a sound processing path 251. The sound processing path 251 comprises a pre-filterbank processing module 254, a filterbank module (filterbank) 256, a post-filterbank processing module 258, a channel selection module 260, and a channel mapping and encoding module 262, each of which are described in greater detail below.

More specifically, the electrical sound signals 203 generated by the input devices 118 are provided to the pre-filterbank processing module 254. The pre-filterbank processing module 254 is configured to, as needed, combine the electrical sound signals 203 received from the input devices 113 and prepare/enhance those signals for subsequent processing. The operations performed by the pre-filterbank processing module 254 may include, for example, microphone directionality operations, noise reduction operations, input mixing operations, input selection/reduction operations, dynamic range control operations and/or other types of signal enhancement operations. The operations at the pre-filterbank processing module 254 generate a pre-filterbank output signal 255, which is also referred to interchangeably herein as “audio signal data 255,” that, as described further below, provides the basis of further sound processing operations. The pre-filterbank output signal 255 represents audio signal data that is a combination (e.g., mixed, selected, etc.) of the input signals (e.g., mixed, selected, etc.) received at the sound input devices 113 at a given point in time.

In operation, the pre-filterbank output signal 255 generated by the pre-filterbank processing module 254 is provided to the filterbank module 256. The filterbank module 256 generates a suitable set of bandwidth limited channels, or frequency bins, that each includes a spectral component of the received sound signals. That is, the filterbank module 256 comprises a plurality of band-pass filters that separate the pre-filterbank output signal 255 into multiple components/channels, each one carrying a frequency sub-band of the original signal (i.e., frequency components of the received sounds signal).

The channels created by the filterbank module 256 are sometimes referred to herein as sound processing channels, and the sound signal components within each of the sound processing channels are sometimes referred to herein as band-pass filtered signals or channelized signals. The band-pass filtered or channelized signals created by the module 256 are processed (e.g., modified/adjusted) as they pass through the sound processing path 251. As such, the band-pass filtered or channelized signals are referred to differently at different stages of the sound processing path 251. However, it will be appreciated that reference herein to a band-pass filtered signal or a channelized signal may refer to the spectral component of the received sound signals at any point within the sound processing path 251 (e.g., pre-processed, processed, selected, etc.).

At the output of the filterbank module 256, the channelized signals are initially referred to herein as pre-processed signals 257. The total number ‘n’ of channels and pre-processed signals 257 generated by the filterbank module 256 may depend on a number of different factors including, but not limited to, implant design, number of active electrodes, coding strategy, and/or recipient preference(s). In certain arrangements, twenty-two (22) channelized signals are created and the sound processing path 251 is said to include 22 channels.

The pre-processed signals 257 are provided to the post-filterbank processing module 258. The post-filterbank processing module 258 is configured to perform a number of sound processing operations on the pre-processed signals 257. These sound processing operations include, for example, channelized gain adjustments for hearing loss compensation (e.g., gain adjustments to one or more discrete frequency ranges of the sound signals), noise reduction operations, speech enhancement operations, etc., in one or more of the channels. After performing the sound processing operations, the post-filterbank processing module 258 outputs a plurality of processed channelized signals 259.

In the specific arrangement of FIG. 2A, the sound processing path 251 includes a channel selection module 260. The channel selection module 260 is configured to perform a channel selection process to select, according to one or more selection rules, which of the ‘m’ channels should be used in hearing compensation. The signals selected at channel selection module 260 are represented in FIG. 2A by arrow 261 and are referred to herein as selected channelized signals or, more simply, selected signals.

In the embodiment of FIG. 2A, the channel selection module 260 selects a subset ‘m’ of the ‘n’ processed channelized signals 259 for use in generation of electrical stimulation signals for delivery to a recipient (i.e., the sound processing channels are reduced from ‘n’ channels to ‘m’ channels). In one specific example, the ‘m’ largest amplitude channels (maxima) from the ‘n’ available channel signals is made, with ‘n’ and ‘m’ being programmable during initial fitting, and/or operation of the prosthesis. It is to be appreciated that different channel selection methods could be used, and are not limited to maxima selection. It is also to be appreciated that, in certain embodiments, the channel selection module 260 may be omitted. For example, certain arrangements may use a continuous interleaved sampling (CIS), CIS-based, or other non-channel selection sound coding strategy.

The sound processing path 251 also comprises the channel mapping module 262. The channel mapping module 262 is configured to map the amplitudes of the selected signals 261 (or the processed channelized signals 259 in embodiments that do not include channel selection) into stimulation signal data (e.g., stimulation commands) that represents electrical stimulation signals that are to be delivered to the recipient so as to evoke perception of at least a portion of the received sound signals. This channel mapping may include, for example, threshold and comfort level mapping, dynamic range adjustments (e.g., compression), volume adjustments, etc., and may encompass selection of various sequential and/or simultaneous stimulation strategies.

As noted, the sound processing path 251 generally operates to convert received sound signals into stimulation control signal data 263 for use in delivering stimulation to the recipient in a manner that evokes perception of the sound signals. In parallel with operations performed for the sound processing path 251, one or more sound signal attributes can also be extracted from the electrical sound signals 203 via sound analysis logic 174 utilizing a sound signal attribute extraction processing path 273. In FIG. 2A, the functional operations enabled by the sound analysis logic 174 (e.g., sound signal attribute extraction operations performed by one or more processors 170 when executing the sound analysis logic 174) are generally represented by attribute extraction pre-processing module 266, attribute extraction module 268, and environmental classifier 264 for the sound signal attribute extraction processing path 273.

During operation, sound signal attributes, such as one or more measures of fundamental frequency (F0) (e.g., frequency or magnitude), Periodic Probability Estimate (PPE) signal attributes, environmental classifier data, etc. may be extracted from the received sound signals and included, along with the stimulation control signal data 263, in one or more data packets 188 wirelessly transmitted to implantable component 112 via wireless communication link 186. In other examples, sound signal attributes may include, but not be limited to, other percepts or sensations (e.g., the first formant (F1) frequency, the second formant (F2) frequency), and/or other formants), other harmonicity measures, rhythmicity measures, measures regarding the static and/or dynamic nature of the sound signals, input volume, etc.

The implantable component 112, utilizing implantable sound processing module 158, can make processing decisions using the sound signal attributes in order to generate electrical stimulation signals for delivery to the recipient. In some instances, the implantable component 112 can use the sound signal attributes together with features extracted from its own sensory inputs, such as, for example, an accelerometer in order to generate stimulation signals for delivery to the recipient.

In one example, the F0 can then be incorporated into the stimulation control signal data 263 at the implantable component 112 in a manner that produces a more salient pitch percept to the recipient. Henceforth the percept of pitch elicited by the acoustic feature F0 in the sounds signals is referred to as “F0-pitch.”

As used herein, a “sensory/sound signal attribute” or “feature” of a received sensory/sound signal refers to an acoustic property of the signal that has a perceptual correlate. For instance, intensity is an acoustic signal property that affects perception of loudness, while the fundamental frequency (F0) of an acoustic signal (or set of signals) is an acoustic property of the signal that affects perception of pitch. In other examples, the signal features may include, for example, other percepts or sensations (e.g., the first formant (F1) frequency, the second formant (F2) frequency), and/or other formants), other harmonicity measures, rhythmicity measures, measures regarding the static and/or dynamic nature of the sound signals, etc. As described further below, these or other sound signal attributes may be extracted and used as the basis for one or more adjustments or manipulations for incorporation into the stimulation signals delivered to the recipient.

In certain examples, the stimulation control signal data 263 may include one or more adjustments (enhancements) that are based on specific sound signal attributes extracted from the received sound signals. That is, the external sound processing module 124 is configured to determine one or more attribute-based adjustments for incorporation into the stimulation control signal data 263, where the one or more attribute-based adjustments are incorporated at one or more points within the sound processing path 251 (e.g., at module 258, etc.). The attribute-based adjustments may take a number of different forms.

In accordance with embodiments presented herein, an element of each of these adjustments is that the adjustments can all be made based on one or more sound signal attributes and, as describe further below, in some instances, the attribute-based adjustments in accordance with embodiments presented herein may be controlled, at least partially, based on an environmental classification of the sound signals.

In order to incorporate attribute-based adjustments into the stimulation control signal data 263, the one or more sound signal attributes that form the basis for the adjustment(s) need to first be extracted from the received sound signals using an attribute extraction process. Certain embodiments presented herein are directed to techniques for controlling/adjusting one or more parameters of the attribute extraction process based on a sound environment of the input sound signals. As described further below, controlling/adjusting one or more parameters of the attribute extraction process based on the sound environment tailors/optimizes the feature extraction process for the current/present sound environment, thereby improving the attribute extraction processing (e.g., increasing the likelihood that the signal features are correctly identified and extracted) and improving the feature-based adjustments, which ultimately improves the stimulation control signal data 263 that is used for generation of stimulation signals for delivery to the recipient.

The fundamental frequency (F0) is the lowest frequency of vibration in a sound signal such as a voiced-vowel in speech or a tone played by a musical instrument (i.e., the rate at which the periodic shape of the signal repeats). In these illustrative examples, an F0-pitch enhancement can be incorporated into sound signal processing, either at the external component 104 or the implantable component 112, such that the amplitudes of the signals in certain channels can be modulated at the F0 frequency, thereby improving the recipient's perception of the F0-pitch. It is to be appreciated that specific reference to the extraction and use of the F0 frequency (and the subsequent F0-pitch enhancement) is merely illustrative and, as such, the techniques presented herein may be implemented to extract other sound signal attributes that can be used to enhance sound signal processing. For example, sound signal attributes may include the F0 harmonic frequencies and magnitudes, PPE signal attributes non-harmonic signal frequencies and magnitudes, environmental classifier data, etc. PPE signal attributes may include estimates of the probability that the input signal in any frequency channel is related to the estimated most dominant F0 and may provide a channel periodic probability signal attribute for each channel.

Returning to the specific example of FIG. 2A, shown is an environmental classifier 264 that is also configured to receive the electrical sound signals 203 generated by the microphone 118A, microphone 118B, and/or auxiliary input 128 (i.e., the electrical sound signals 203 are directed to both the pre-filterbank processing module 254 and the environmental classifier 264). The environmental classifier 264 represents additional functional operations that may be enabled by the sound analysis logic 174 of FIG. 1D (i.e., additional operations that may be performed by the one or more processors 170 when executing the sound analysis logic 174). The environmental classifier 264 is configured to “classify” or “categorize” the sound environment of the sound signals received at the input devices 113. The environmental classifier 264 may be configured to categorize the sound environment into a number of classes/categories. In one illustrative example, the environmental classifier 264 may be configured to categorize the sound environment into one of six (6) categories, including “Speech,” “Noise,” “Speech in Noise,” “Music,” “Wind,” and “Quiet.” In general, the environmental classifier 264 operates to determine a category of the sound signals using a type of decision structure (e.g., decision tree, alternative machine learning designs/approaches, and/or other structures that operate based on individual extracted characteristics from the input signals). Sound signal attributes output by the environmental classifier, as generally represented by arrow 265 (also referred to herein as “environmental classifier data 265”), can include, but not be limited to, classifications or categorizations or probabilities of the sound environment, which can be provided to packet logic for inclusion in one or more data packets 188 wirelessly transmitted to implantable component 112.

In some instances, the category of the sound environment associated with the sound signals may be used to adjust one or more settings/parameters of the sound processing path 251 for different listening situations or environments encountered by the recipient, such as noisy or quiet environments, windy environments, or other uncontrolled noise environments. In FIG. 2A, arrows 265 generally illustrate that different operating parameters of one or more of the modules 254, 256, 258, 260, and/or 262 could be adjusted for operation in different sound environments.

In some embodiments, the extracted sound signal attributes 269 may also be used by the external sound processing module 124 to incorporate one or more signal adjustments at one or more of modules 254, 256, 258, 260, and/or 262 of the sound processing path 251, as generally illustrated by dashed-line arrows 269. In some embodiments, the attribute extraction pre-processing module 266, attribute extraction module 268, and attribute adjustment module 270 collectively form a sound signal attribute extraction processing path 273 that is separate from, and runs in parallel to, the sound processing path 251. As such, the attribute extraction pre-processing module 266 separately receives the electrical sound signals 203 generated by the microphone 118A, microphone 118B, and/or auxiliary input 128. Although sound signal attribute extraction processing path 273 is illustrated as a separate processing path in FIG. 2A, in some embodiments, one or more sound signal attributes could also be extracted via one or more attribute extraction modules 268, that may receive input data output from each of modules 254, 256, 258, and/or 260 along the sound processing path 251.

The attribute extraction pre-processing module 266 may be configured to perform operations that are similar to the pre-filterbank processing module 254. For example, the attribute extraction pre-processing module 266 may be configured to perform microphone directionality operations, noise reduction operations, input mixing operations, input selection/reduction operations, dynamic range control operations, and/or other types of signal enhancement operations to generate a pre-processing signal, generally represented in FIG. 2A by arrow 267. The attribute extraction module 268 performs one or more operations to extract one or more target/desired sound signal attribute(s) from the pre-processed signal (e.g., F0 estimate, etc.). The attribute extraction module 268 may include Periodic Probability Estimate (PPE) logic (not shown), which may derive PPE signal attributes from the electrical sound signals 203, such as a periodic probability value for the electrical sound signals 203 by compression limiting and smoothing the signals representative or the ratio of the power related to the most dominant fundamental frequency to the total signal power present in the electrical sound signals 203. In some instances, PPE logic provided for the attribute extraction module 268 may further estimate the probability that the signal in any frequency channel is related to the estimated most dominant fundamental frequency of the electrical sound signals in order to generate a channel periodic probability signal attribute for each channel using the frequency and power of any sinusoidal frequency components present in the electrical sound signals determined from F0 estimates.

As represented by arrow 269, the extracted sound signal attribute(s) extracted by the attribute extraction module 268 are provided to packet logic 176 and, for embodiments in which attribute adjustments are to be implemented by external sound processing module 124, can also be provided to one or more of module(s) 254, 256, 258, 260, and/or 262 for incorporating attribute-based adjustments into the sound processing path 251 and, accordingly, into the stimulation control signal data 263.

In summary, FIG. 2A illustrates an embodiment in which two processing paths operate in parallel. The first processing path, referred to above as the sound processing path 251, uses sound processing operations to convert the received sound signals in stimulation control signal data 263. The sound processing operations of the sound processing path 251 can be controlled/adjusted so as to optimize the recipient's perception of the sound signals received via the input devices 113. In contrast, the second processing path, referred to above as the sound signal attribute extraction processing path 273, includes sound signal attribute extraction operations that are controlled/selected so as to extract one or more extracted sound signal attributes, as represented by arrow 269, from the sound signals received via the input devices 113 and, in some embodiments, to incorporate a attribute-based adjustment(s) into the stimulation control signal data 263. In addition to extracted sound signal attribute(s) 269 that can be extracted via the sound signal attribute extraction processing path 273, sound signal attribute(s) including environmental classifications/categorizations of the sound environment regarding the input sound signals, as represented by environmental classifier data 265, can also be provided via environmental classifier 264. Accordingly, as referred to herein, sound signal attributes that can be generated by external sound processing module 124 can include any combination of extracted sound signal attributes 269 and/or environmental classifier data 265.

Further understanding of the embodiment of FIG. 2A may be appreciated through the description of several illustrative use cases relating to determining an environmental classification of a sound environment. For example, consider a situation in which a tonal language (e.g., Mandarin) speaking recipient is located in a noisy café in China and is attempting to converse with another individual. In this example, the environmental classifier 264 determines that recipient is located in a “Speech in Noise” environment (i.e., the sound signals detected by the microphones 118A and 118B are “Speech in Noise” signals). As result of this determination, the environment classifier 264 can configure or provide environmental classifier data 265 that facilitates adjusting the pre-filterbank processing module 254 to perform microphone beamforming to optimize listening from directly in front of the recipient, and to perform Background Noise Reduction.

Now consider a recipient attending a music concert. In this example, the environmental classifier 264 determines that recipient is located in a “Music” environment. As result of this determination, the environmental classifier 264 can configure or provide environmental classifier data 265 that facilitates adjusting the pre-filterbank processing module 254 to a “Moderate” microphone directionality, which is only partly directional allowing for sound input from a broad area ahead of the recipient.

Returning to features of FIG. 2A, for embodiments in which attribute adjustments are not incorporated along sound processing path 251 or for embodiments in which additional attribute adjustments may be desired at the implantable component 112, the one or more extracted sound signal attributes 269 output by attribute extraction module 268 and/or environmental classifier data 265 output by environmental classifier 264 can be provided to packet logic 176 that generates one or more data packets 188 in which at least one packet includes both the one or more sound signal attributes (any combination of extracted sound signal attributes 269 and/or environmental classifier data 265) along with the corresponding stimulation control signal data 263.

In various embodiments, attribute extraction module 268 and environmental classifier 264 can be configured to generate sound signal attributes at any time, such as at specific points in time in conjunction with the input sound signal, for example at regular intervals (e.g., 10 times a second), at changes in the input sound signal (e.g., when a transition occurs), or based on any other rule or setting that can be used for correlating sound signal attributes with the input sound signal and the resultant stimulation control signal data 263 generated via the sound processing path 251 or audio signal data, as discussed below.

Packet logic 176 generates a stream of data packets 188, such that the sound signal attributes (265, 269) generated via attribute extraction module 268 and environmental classifier 264 are correlated in time with the stimulation control signal data 263 (sensory/sound data) generated via the sound processing path 151. The time alignment could be performed using any technique. For example, the packet logic 176 can packetize the stimulation control signal data 263 into stimulation control signal data samples within data packets 188 in which the data samples can be time-aligned with extracted sound signal attributes 269 and/or environmental classifier data 265, if present (recall, sensory/sound signal attributes can be periodically determined) that can also be included in one or more of the data packets 188. The stream of data packets 188 are wirelessly transmitted to the implantable component via wireless transceiver 120.

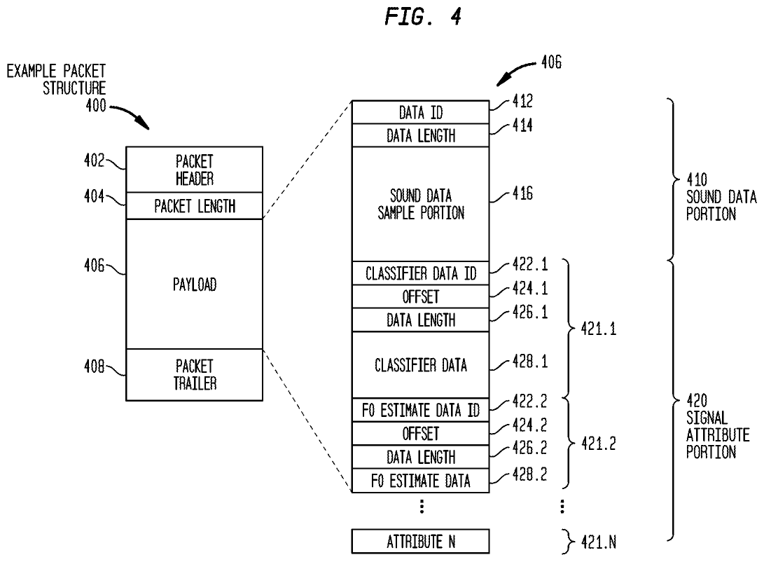

The data samples and the time-aligned sound signal attributes, if present, that can be included in one or more of data packets 188 can be clearly defined within the packets in order for the implantable component 112 to separate the sound signal attributes and the data samples into independent streams that are clearly correlated in time so that the implantable component 112 can sensibly use the information contained in the data packets 188 for further sound processing or other functionality that may be performed via implantable sound processing module 158. For example, the implantable component 112, via implantable sound processing module 158 can use sound signal attributes such as F0 estimates to provide the OPAL processing strategy with F0, or harmonic probabilities that can be used to generate stimulation control signals for delivery to the recipient. FIG. 4, discussed in further below, provides additional details regarding the potential structure of one or more packets that can be utilized to wirelessly communicate sound data and sound signal attributes to an implantable component, such as implantable component 112.

With reference to FIG. 2B, FIG. 2B is a functional block diagram illustrating further details of the implantable component 112 of the cochlear implant system 102 configured to implement certain techniques in which implantable sound processing module 158 generates electrical stimulation signals (e.g., current signals) utilizing the stimulation control signal data 263 (sensory/sound data) included in the one or more data packets 188 and sound signal attributes, if present, that can also be included in one or more of the data packets 188 received by wireless transceiver 180 via wireless communications from wireless transceiver 120 of the external component 104. In FIG. 2B, the functional operations enabled by the implantable sound processing logic 190 (i.e., the operations performed by the one or more processors of implantable sound processing module 158 when executing the sound processing logic 190) are generally represented by signal adjustment module 194.

During operation for the one or more data packets 188 received by wireless transceiver 180, packet logic 192 (e.g., performed by the one or more processors when executing the packet logic 192), de-maps or otherwise separates the stimulation control signal data 263 and the time-aligned sound signal attributes (i.e., 269/265) from the data packets 188. Packet logic 192 provides the stimulation control signal data 263 and the time-aligned sound signal attributes (i.e., 269/265) to the signal adjustment module 194 for further processing and the generation of stimulation control signals 195 that are utilized via stimulator unit 142 for generating electrical stimulation signals for delivery to the recipient via stimulating assembly 116. For example, environmental classifier data 265 may be used to enable or disable channels and/or enable noise reduction features in the channel domain at the implantable component 112. In another example, as noted above, a sound signal attribute such as F0 estimate can be used in the case of OPAL processing in order to add envelope modulation back into the channel data at the implantable component 112 in order to add a strong F0 cue for the recipient to hear. In yet another example, the accuracy of an F0 estimate is aided if there is an additional measure of the “probability” of the harmonics being directly related to the estimated F0, which can be provided via a PPE signal attribute. If the probability is high, then the F0 is likely to be an accurate estimate, compared to if the probability is low. Thus, in some instances, sound signal attributes can include a PPE signal attribute, such as a harmonic probability value, that can be sent to the implantable component 112 in addition to an F0 estimate value.

In some instances, a Periodic Probability Estimate (PPE) can also be calculated in one or more frequency channels of interest. This could be in ⅓ octave bands, or more typically one or more of the stimulation channels that are used to determine the stimulation control signal data sent to the implant, for example, the 22 channels generated by the filterbank module 256, when n=22. In this case, ‘n’ individual PPE values can be sent to the implant as an array of probability values, along with the F0. The PPE values, when calculated for each channel, indicate the relative probability that the energy in that channel is directly related to the F0 extracted, i.e., if it contains one of the harmonics of the F0. In the implantable component 112, the PPE values for each channel could then be used to determine how much modulation is applied as per the OPAL coding strategy. For channels with a high probability that the energy is related to the F0, more modulation could then be applied than channels with a low probability that the energy is related to the extracted F0. Thus, in some instances, sound signal attributes sent to the implantable component can include one or more harmonic probability values associated with one or more frequency channels of interest determined from the sound signals.

It is to be understood that any processing of the stimulation control signal data 263 based on the sound signal attributes (269/265) for generating stimulation control signals 195 that are utilized via stimulator unit 142 for generating electrical stimulation signals for delivery to the recipient via stimulating assembly 116.

Although sound signal attributes may not be included in every packet transmitted from the external component 104, the implantable component 112 can process all sound data received from the external component 104 utilizing sound signal attributes received in one or more of data packets 188 received from the external component 104. For example, a first received packet may include sound data and one or more sound signal attributes that can be utilized by the implantable component in processing the sound data included in the first packet, as well as any subsequent packet that is received by the implantable component 112 but for which no sound signal attribute(s) are included in the subsequent packets, for example, if the sound signal attribute(s) have not been updated/changed since receiving the first packet. Thus, even if sound signal attribute(s) are not included in all packets received by the implantable component 112, processing of the sound data can still be performed using sound signal attribute(s) included in one or more previously received packets.

Turning to FIGS. 3A-3B, FIG. 3A is a functional block diagram illustrating another example arrangement of the sound processing unit 106 of the external component 104 of the cochlear implant system 102 and FIG. 3B is a functional block diagram illustrating another arrangement of the implantable component 112 of the cochlear implant system 102 according to an example embodiment. In contrast to FIG. 2A and 2B, for the embodiment of FIGS. 3A and 3B audio signal data 255 generated via pre-filterbank processing module 254 is packetized via packet logic 176 along with time-aligned sound signal attributes 269 (e.g., F0 estimates) generated via attribute extraction module 268 and sound signal attributes, such as environmental classifier data 265 generated via environmental classifier 264 to generate data packets 188, which are transmitted to the implantable component 112 via wireless transceiver 120. In some instances, the audio signal data 255 may include directional microphone, level adjustment, and/or noise reduction processing.

Thereafter, data packets 188 received at the implantable component 112 via wireless transceiver 180 are de-packetized or separated into the audio signal data 255 and the sound signal attributes (269/265), if present, in which the audio signal data 255 can be further processed based on the sound signal attributes by the implantable sound processing module 158 via sound processing logic 190, which, for the embodiment of FIG. 3B, includes the filterbank module 256, the post-filterbank processing module 258, the channel selection module 260, and the mapping and encoding module 262. Modules 256, 258, 260, and 262 may perform operations similar to those as discussed above for FIG. 2A, except that for the embodiment of FIG. 3B it is assumed that encoding module 262 generates stimulation control signals 195 that are utilized via stimulator unit 142 for generating electrical stimulation signals for delivery to the recipient via stimulating assembly 116.

The embodiments of FIGS. 2A-2B and 3A-3B only illustrate two of the many potential sensory/sound processing functionality splits that can be utilized according to techniques of the present disclosure in order to wirelessly communicate sensory/sound data and sensory/sound signal attributes from an external component to an internal component of a medical device. It is to be appreciated that virtually any split of sound processing functionality can be envisioned by embodiments herein in order to wirelessly communicate sensory/sound data and sensory/sound signal attributes from an external component to an internal component of a medical device and, thus, are clearly within the scope of the teachings of the present disclosure.

One key benefit of the techniques herein involving wireless communications from an external component to an implantable component of a cochlear implant system that include sound data and sound signal attributes associated with the sound data is the potential saving of unnecessary implant processing (e.g., battery power and longevity). For example, for an implementation of OPAL in a system that is split between external and internal components, with an audio or stimulation control signal in-between, it can be much more costly, in terms of power consumption, to implement F0 processing in the implant. Thus, techniques herein offer improvements over conventional cochlear implant systems, in terms of potential implant power consumption, by providing for the ability to move complex signal processing operations out of the implant and into the external component, such that sound signal attributes and sound data can be wirelessly communicated to the implant for minimal processing using the signal attributes (e.g., channel manipulation, etc.) in order to generate electrical stimulation signals for delivery to a recipient.

Turning to FIG. 4, FIG. 4 is a schematic diagram illustrating example details for a packet structure 400 that can be utilized in accordance with techniques herein to combine sensory/sound data along with one or more sensory/sound signal attributes into one or more packets (e.g., data packets 188) that can be communicated from an external component to an implantable component of a cochlear implant system, such as from external component 104 to implantable component 112 of cochlear implant system 102.

As shown in FIG. 4, the packet structure 400 may include a packet header portion 402, a packet length field 404, a payload portion 406, and a packet trailer portion 408. In various embodiments, the packet header portion 402 may include any suitable field that may be used to facilitate wireless communications between one or more entities, such as address information (e.g., Internet Protocol (IP) address information, Media Access Control (MAC) address information), port information, interface information, version information, etc. The packet length field 404 may identify the overall length of a given packet (e.g., in bytes). The payload portion 406 may include a sound data portion 410 and an optional a sound signal attribute portion 420, discussed in further detail below. In various embodiments, the packet trailer portion 408 can carry information that can be utilized for error correction, such as a Cyclic Redundancy Check (CRC) value, a Forward Error Correction (FEC) value, a checksum value, or the like. It is to be understood that any sound data and sound signal attribute information discussed with reference to FIG. 4 may be inclusive of any sensory data and sensory signal attribute information, in accordance with embodiments herein.

The sound data portion 410 of the packet structure 400 may include a data identifier (ID) portion 412, a data length field 414, and a sound data portion 416 in which the sound data portion 416 may be of variable length, depending on the amount of sound data (e.g., samples) carried in a given packet. The data ID portion 412 may carry information used to identify or confirm an order of the sound data carried in the sound data portion 416, such as a sequence number or the like. The data length field 414 can identify a length of sound data carried in the sound data portion 416. In one example, the data length field 414 can be set to a value indicating the number of sound data samples carried in the sound data portion 416. The sound data portion 416 can include a variable number of samples of sound data, such as a variable number of samples of audio signal data (i.e., audio signal data 255) or stimulation control signal data (i.e., stimulation control signal data 263) as discussed herein.

The sound signal attribute portion 420 of the packet structure 400 may be optional, as not all packets transmitted from the external component may include one or more signal attributes. Recall, as discussed above, that attribute extraction module 268 and environmental classifier can be configured to generate sound signal attributes at an time, such as at specific points in time in conjunction with the input sound signal, for example, at regular intervals (e.g., 10 times a second), at changes in the input sound signal (e.g., when a transition occurs), or based on any other rule or setting that can be used for correlating sound signal attributes with the input sound signal with resultant audio signal data or stimulation control signal data. As such, some packets may include only a signal data portion 410.

Regarding the packet structure 400 for packet(s) including sound signal attributes, the sound signal attribute portion 420 can include an ‘N’ number of sound signal attribute data blocks 421 (e.g., blocks 421.1-421.N, as shown in FIG. 4). Each sound signal attribute data block 421 may include an attribute data ID field 422, an offset field 424, a data length field 426, and an attribute data portion 428.

The attribute data ID field 422 can identify the type of sound signal attribute data carried in a given sound signal attribute data block 421 (e.g., F0 estimate, PPE signal attribute, environmental classifier data, etc.). In one instance, each sound signal attribute data type that may be included in one or more packet(s) can be set to a corresponding predefined value to facilitate proper identification of each signal attribute data type. To facilitate time-alignment between the sound data samples carried in the sound data portion 410 and at least one sound signal attribute carried in a given sound signal attribute data block 421, the offset field 424 can identify a sample number at which a corresponding sound signal attribute is to be applied to the sound data carried in the sound data portion of a given packet and the data length field 426 can identify the length (e.g., in bits or bytes) of the sound signal attribute data carried in the attribute data portion 428, which can include a given sound signal attribute included in a given packet.

For example, as illustrated in FIG. 4, consider that a first sound signal attribute data block 421.1 carries environmental classifier data such that the attribute data ID field 422.1 is set to a type “Classifier Data” that identifies that environmental classifier data is carried in the attribute data portion 428.1. In another example, as illustrated in FIG. 4, consider that a second sound signal attribute data block 421.2 carries an F0 estimate such that the attribute data ID field 422.2 is set to a type “F0 Estimate Data” that identifies that an F0 estimate is carried in the attribute data portion 428.2.

Use of the offset field 424 to identify the sample of sound data at which a given sound signal attribute carried in a given sound signal attribute data block 421 is to be applied may be varied. Consider one example in which signal data carried in the sound signal data portion 416 is 16 samples in length and the F0 estimate included in the attribute data portion 428.2 second sound signal attribute data block 421.2 is to be changed 8 samples into the signal data. In this example, the offset field 424.2 could be set to a value of “8” to indicate that the F0 estimate is to be applied starting at the eighth sound data sample of a packet. Consider another example in which an input volume signal attribute (not shown in FIG. 4) is to be changed from a value of “4” to “6” at the start of an audio frame. In this example the offset field for such a sound signal attribute data block could be set to a value of “0” or “1” (depending on the relative sample numbering scheme used for packets) to indicate that the input volume is to be applied starting at the first sound data sample of a packet. Other variations involving the offset scheme can be envisioned.

For instances in which a given sound signal attribute is not changed for a given packet of sound data, the sound signal attribute can be omitted from the sound signal attribute portion of the packet. For example, consider an instance in which environmental classifier data for a set of sound data samples is unchanged from a previous setting applied to a previous number of sound data samples. In this instance, environmental classifier data can be omitted from the sound signal attribute portion of the packet altogether.

In various embodiments, encryption and/or compression could be applied to one or more portions of packets, such as to one or both of the sound data portion 410 and/or the sound signal attribute portion 420 (if provided) of a packet.

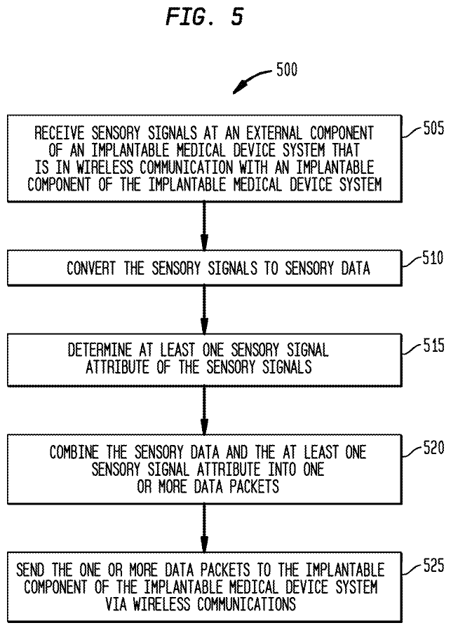

With reference now made to FIG. 5, depicted therein is a flowchart 500 illustrating a process flow for implementing the techniques of the present disclosure. Flowchart 500 begins in operation 505 where sensory signals (e.g., sound signals) are received at an external component of an implantable medical device system (e.g., an implantable hearing device system) that is in wireless communication with an implantable component of the implantable medical device system. At 510, the external component converts the sensory signals to sensory data (e.g., audio signal data or stimulation control signal data, as discussed herein). At 515, the external component determines at least one sensory signal attribute of the sensory signals (e.g., an extracted sound signal attribute, environmental classifier data, etc., as discussed herein). At 520, the external component combines the sensory data and the at least one sensory signal attribute into one or more data packets. At 525, the external component sends the one or more data packets to the implantable component of the implantable medical device system via wireless communications

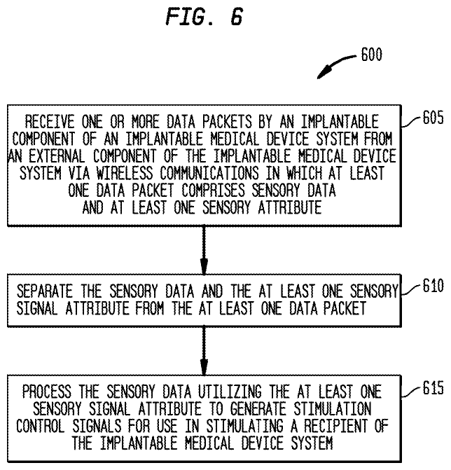

With reference now made to FIG. 6, depicted therein is a flowchart 600 illustrating a process flow for implementing the techniques of the present disclosure. Flowchart 600 begins in operation 605 where an implantable component of an implantable medical device system (e.g., an implantable hearing device system) receives one or more data packets from an external component of the implantable medical device system via wireless communications, wherein at least one data packet comprises sensory data (e.g., sound data, such as audio signal data or stimulation control signal data, as discussed herein) and at least one sensory signal attribute (e.g., an extracted sound signal attribute, environmental classifier data, etc., as discussed herein). At 610, the implantable component separates the sensory data and the at least one sensory signal attribute from the at least one data packet. At 615, the implantable component processes the sensory data utilizing the at least one sensory signal attribute to generate stimulation control signals for use in stimulating a recipient of the implantable medical device system.

Accordingly, the method of flowchart 500 provides for a process in which the external component can combine sensory/sound data (such as audio signal data or stimulation control signal data that has been generated/processed by the external component from sound signals received by the external component) with one or more sensory/sound signal attributes determined from input sensory/sound signals into one or more packets that can be wirelessly transmitted to the implantable component. Further, the method of flowchart 600 provides a process in which the implantable component can use the sensory/sound data and one or more sensory/sound signal attributes to generate electrical stimulation signals for delivery to a recipient of the implantable hearing device system.

In addition to the features described above with reference to FIGS. 2A, 2B, 3A, 3B and 4-6, the techniques of the present disclosure may be used to drive the functionality of additional features of hearing devices.

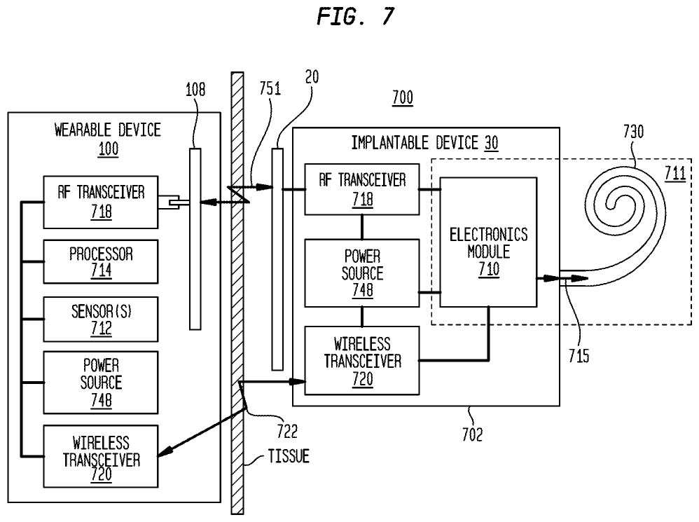

As previously described, the technology disclosed herein can be applied in any of a variety of circumstances and with a variety of different devices. Example devices that can benefit from technology disclosed herein are described in more detail in FIG. 7, below. As described below, the operating parameters for the devices described with reference to FIG. 7 may be configured according to the techniques described herein. The techniques of the present disclosure can be applied to other medical devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue, to the extent that the operating parameters of such devices may be tailored based upon the posture of the recipient receiving the device. Further, technology described herein can also be applied to consumer devices. These different systems and devices can benefit from the technology described herein. For example, the data and signal attribute wireless transmission techniques of the present disclosure may be applied to consumer grade or commercial grade headphone or ear bud products.

FIG. 7 is a functional block diagram of an implantable stimulator system 700 that can benefit from the technologies described herein. The implantable stimulator system 700 includes a wearable device 100 acting as an external processor device and an implantable device 30 acting as an implanted stimulator device. In examples, the implantable device 30 is an implantable stimulator device configured to be implanted beneath a recipient's tissue (e.g., skin). In examples, the implantable device 30 includes a biocompatible implantable housing 702. Here, the wearable device 100 is configured to transcutaneously couple with the implantable device 30 via a wireless connection to provide additional functionality to the implantable device 30.

In the illustrated example, the wearable device 100 includes one or more sensors 712, a processor 714, an RF transceiver 718, a wireless transceiver 720, and a power source 748. The one or more sensors 712 can be one or more units configured to produce data based on sensed activities. In an example where the stimulation system 700 is an auditory prosthesis system, the one or more sensors 712 include sound input sensors, such as a microphone, an electrical input for an FM hearing system, other components for receiving sensory/sound input, or combinations thereof. Where the stimulation system 700 is a visual prosthesis system, the one or more sensors 712 can include one or more cameras or other visual sensors. Where the stimulation system 700 is a cardiac stimulator, the one or more sensors 712 can include cardiac monitors. The processor 714 can be a component (e.g., a central processing unit) configured to control stimulation provided by the implantable device 30. The stimulation can be controlled based on data from the sensor 712, a stimulation schedule, or other data. Where the stimulation system 700 is an auditory prosthesis, the processor 714 can be configured to convert sound signals received from the sensor(s) 712 (e.g., acting as a sound input unit) into signals 751. The RF transceiver 718 is configured to send the signals 751 in the form of power signals, data signals, combinations thereof (e.g., by interleaving the signals), or other signals. The RF transceiver 718 can also be configured to receive power or data. Stimulation control signals can be generated by the processor 714 and transmitted, using the RF transceiver 718, to the implantable device 30 for use in providing stimulation.

Where the stimulation system 700 is an auditory prosthesis configured to facilitate wireless communications involving one or more data packets that can include sensory/sound data and at least one data packet can include sensory/sound data and at least one sensory/sound signal attribute of input sound signals, the processor 714 can be configured via packet logic configured for the wearable device 100 (e.g., packet logic 176, as shown in FIG. 1D) to convert sensory/sound signals received from the sensor(s) 712 (e.g., acting as a sound input unit) into one or more data packets that can be wirelessly communicated to the implantable device 30 via a wireless communication link 722 facilitated via wireless transceivers 720. The wireless transceiver 720 is configured to send the data packets that can include sensory/sound data (e.g., stimulation control signal data or audio signal data) and, for instances in which one or more sensory/sound signal attribute(s) are to be included in the packets, the one or more sensory/sound signal attribute(s) can be included in the packets in a time-aligned manner, in order to be applied to the sensory/sound data starting at a given sample of the sensory/sound data as identified via information included in the packets.

In the illustrated example, the implantable device 30 includes an RF transceiver 718, a wireless transceiver 720, a power source 748, and a medical instrument 711 that includes an electronics module 710 and a stimulator assembly 730. The implantable device 30 further includes a hermetically sealed, biocompatible implantable housing 702 enclosing one or more of the components.

The electronics module 710 can include one or more other components to provide medical device functionality. In many examples, the electronics module 710 includes one or more components for receiving a signal and converting the signal into the stimulation signal 715. The electronics module 710 can further include a stimulator unit. The electronics module 710 can generate or control delivery of the stimulation signals 715 to the stimulator assembly 730. In examples, the electronics module 710 includes one or more processors (e.g., central processing units or microcontrollers) coupled to memory components (e.g., flash memory) storing instructions that when executed cause performance of an operation. In examples, the electronics module 710 generates and monitors parameters associated with generating and delivering the stimulus (e.g., output voltage, output current, or line impedance). The stimulator assembly 730 can be a component configured to provide stimulation to target tissue. In the illustrated example, the stimulator assembly 730 is an electrode assembly that includes an array of electrode contacts disposed on a lead. The lead can be disposed proximate tissue to be stimulated. Where the system 700 is a cochlear implant system, the stimulator assembly 730 can be inserted into the recipient's cochlea. The stimulator assembly 730 can be configured to deliver stimulation signals 715 (e.g., electrical stimulation signals) generated by the electronics module 710 to the cochlea to cause the recipient to experience a hearing percept. In other examples, the stimulator assembly 730 is a vibratory actuator disposed inside or outside of a housing of the implantable device 30 and configured to generate vibrations. The vibratory actuator receives the stimulation signals 715 and, based thereon, generates a mechanical output force in the form of vibrations. The actuator can deliver the vibrations to the skull of the recipient in a manner that produces motion or vibration of the recipient's skull, thereby causing a hearing percept by activating the hair cells in the recipient's cochlea via cochlea fluid motion.

The RF transceivers 718 can be components configured to transcutaneously receive and/or transmit a signal 751 (e.g., a power signal and/or a data signal). The RF transceiver 718 can be a collection of one or more components that form part of a transcutaneous energy or data transfer system to transfer the signal 751 between the wearable device 100 and the implantable device 30. Various types of signal transfer, such as electromagnetic, capacitive, and inductive transfer, can be used to usably receive or transmit the signal 751. The RF transceiver 718 for implantable device 30 can include or be electrically connected to a coil 20.

As illustrated, the wearable device 100 includes a coil 108 for transcutaneous transfer of signals with the concave coil 20. As noted above, the transcutaneous transfer of signals between coil 108 and the coil 20 can include the transfer of power and/or data from the coil 108 to the coil 20 and/or the transfer of data from coil 20 to the coil 108. The power source 748 can be one or more components configured to provide operational power to other components. The power source 748 can be or include one or more rechargeable batteries. Power for the batteries can be received from a source and stored in the battery. The power can then be distributed to the other components as needed for operation.

Regarding wireless transceiver 720 of implantable device 30, sensory/sound data (e.g., stimulation control signal data or audio signal data) and one or more sensory/sound signal attributes can be received by the implantable device 30 via one or more data packets received via wireless transceiver 720. The electronics module 710 may include one or more processor(s) (e.g., central processor unit(s)) that can be configured via packet logic configured for the implantable device 30 (e.g., packet logic 192, as shown in FIG. 1D) to separate sensory/sound data and the one or more sensory/sound signal attributes from the received data packets in order to process the sensory/sound data using the one or more sensory/sound signal attributes for generating simulation signals for delivery to the recipient.

As should be appreciated, while particular components are described in conjunction with FIG. 7, technology disclosed herein can be applied in any of a variety of circumstances. The above discussion is not meant to suggest that the disclosed techniques are only suitable for implementation within systems akin to that illustrated in and described with respect to FIG. 7. In general, additional configurations can be used to practice the methods and systems herein and/or some aspects described can be excluded without departing from the methods and systems disclosed herein.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

1. A method, comprising:
receiving sensory signals at an external component of an implantable medical device system that is in wireless communication with an implantable component of the implantable medical device system;
converting the sensory signals to sensory data;
determining at least one sensory signal attribute of the sensory signals;
combining the sensory data and the at least one sensory signal attribute into one or more data packets; and
sending the one or more data packets to the implantable component of the implantable medical device system via wireless communications.

2. The method of claim 1, wherein the sensory data is sound data, the at least one sensory signal attribute is at least one sound signal attribute, and at least one data packet of the one or more data packets comprises a sound data portion that comprises a plurality of samples of the sound data and a sound signal attribute portion that includes the at least one sound signal attribute.

3. The method of claim 2, wherein the sound data portion further comprises at least one of a data identifier field or a data length field.

4. The method of claim 3, wherein the sound signal attribute portion comprises at least one of an attribute identifier field, an attribute length field, or an offset field and further comprises the at least one sound signal attribute.

5. The method of claim 4, wherein the offset field includes an offset value that identifies a sample of the plurality of samples with which the at least one sound signal attribute is associated.

6. The method of claim 1, wherein the at least one sensory signal attribute is at least one of:
a fundamental frequency (F0) extracted from the sensory signals;
an environmental classification of a sound environment of the sensory signals;
a harmonic probability value associated with an accuracy of a fundamental frequency (F0) extracted from the sensory signals; or
one or more harmonic probability values associated with one or more frequency channels of interest determined from the sensory signals.

7. The method of claim 1, wherein the sensory data includes audio signal data.

8. The method of claim 1, wherein the sensory data includes stimulation control signal data.

9. (canceled)

10. (canceled)

11. (canceled)

12. (canceled)

13. (canceled)

14. (canceled)

15. (canceled)

16. (canceled)

17. One or more non-transitory computer readable storage media comprising instructions that, when executed by a processor, cause the processor to:
convert sound signals received at an external component of an implantable hearing device system to sound data;
determine at least one sound signal attribute of the sound signals; and
stream one or more data packets to an implantable component of the implantable hearing device system via wireless communications, wherein at least one data packet of the one or more data packets comprises the sound data and the at least one sound signal attribute.

18. The non-transitory computer readable storage media of claim 17, wherein the at least one data packet of the one or more data packets comprises a sound data portion that comprises a plurality of samples of the sound data and a sound signal attribute portion that includes the at least one sound signal attribute.

19. The non-transitory computer readable storage media of claim 18, wherein the sound data portion further comprises at least one of a data identifier field or a data length field.

20. The non-transitory computer readable storage media of claim 19, wherein the sound signal attribute portion comprises at least one of an attribute identifier field, an attribute length field, or an offset field and further comprises the at least one sound signal attribute.

21. The non-transitory computer readable storage media of claim 20, wherein the offset field includes an offset value that identifies a sample of the plurality of samples with which the at least one sound signal attribute is associated.

22. The non-transitory computer readable storage media of claim 17, wherein the at least one sound signal attribute is at least one of:
a fundamental frequency (F0) extracted from the sound signals;
an environmental classification of a sound environment of the sound signals;
a harmonic probability value associated with an accuracy of a fundamental frequency (F0) extracted from the sound signals; or
one or more harmonic probability values associated with one or more frequency channels of interest determined from the sound signals.

23. The non-transitory computer readable storage media of claim 17, wherein the sound data includes audio signal data.

24. The non-transitory computer readable storage media of claim 17, wherein the sound data includes stimulation control signal data.

25. An implantable hearing device system comprising:
an external component comprising:
one or more input devices;
a wireless transceiver; and
one or more processors, wherein the one or more processors are configured to:
convert sound signals received at the one or more input devices to sound data;
determine at least one sound signal attribute of the sound signals; and
stream one or more data packets to an implantable component of the implantable hearing device system via wireless communications, wherein at least one data packet of the one or more data packets comprises the sound data and the at least one sound signal attribute.

26. The implantable hearing device system of claim 25, wherein the at least one data packet of the one or more data packets comprises a sound data portion that comprises a plurality of samples of the sound data and a sound signal attribute portion that includes the at least one sound signal attribute.

27. The implantable hearing device system of claim 26, wherein the sound data portion further comprises at least one of a data identifier field or a data length field.

28. The implantable hearing device system of claim 27, wherein the sound signal attribute portion comprises at least one of an attribute identifier field, an attribute length field, or an offset field and further comprises the at least one sound signal attribute.

29. The implantable hearing device system of claim 28, wherein the offset field includes an offset value that identifies a sample of the plurality of samples with which the at least one sound signal attribute is associated.

30. The implantable hearing device system of claim 25, wherein the at least one sound signal attribute is at least one of:
a fundamental frequency (F0) extracted from the sound signals;
an environmental classification of a sound environment of the sound signals;
a harmonic probability value associated with an accuracy of a fundamental frequency (F0) extracted from the sound signals; or one or more harmonic probability values associated with one or more frequency channels of interest determined from the sound signals.
